(19) United States
(12) Patent Application Publication
(10) Pub. No.: US 2023/0364421 A1
(43) Pub. Date: Nov. 16, 2023

(54) PARAMETER OPTIMIZATION BASED ON DIFFERENT DEGREES OF FOCUSING

(71) Applicant: Cochlear Limited, Macquarie University, NSW (AU)

(72) Inventors: Naomi CROGHAN, Centennial, CO (US); Sara Ingrid DURAN, Denver, CO (US); Christopher Joseph LONG, Centennial, CO (US)

(21) Appl. No.: 18/044,905

(22) PCT Filed: Sep. 2, 2021
(86) PCT No.: PCT/IB2021/058015

§ 371 Date: Mar. 10, 2023

Related U.S. Application Data
(60) Provisional application No. 63/080,257, filed on Sep. 18, 2020.

(51) Int. Cl. A61N 1/36

(52) U.S. Cl. CPC A61N 1/36039

(57) ABSTRACT

Presented herein are techniques for setting operational parameters of an implantable medical device system based on a combination of at least one monopolar stimulation measurement and one or more multipolar (focused) stimulation measurements. In particular, the techniques presented herein perform at least one objective or behavioral measurement using monopolar stimulation, and the same objective or behavioral measurement using focused stimulation with one or more degrees of focusing. Hearing outcomes/responses evoked in response to each of the measurements are captured/recorded and evaluated relative to one another to set one or more operational parameters, such as the degree of focusing, of the implantable medical device or implantable medical device system.

### BACKGROUND

### Field of the Invention

The present invention relates generally to setting operational parameters of implantable medical devices.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: performing at least one monopolar stimulation measurement on a recipient of an implantable medical device; performing one or more focused stimulation measurements on the recipient of the implantable medical device, wherein the at least one monopolar stimulation measurement and the one or more focused stimulation measurements obtain the same type of response from the recipient; and determining one or more operational parameters of the implantable medical device based on the responses evoked by at least one monopolar stimulation measurement and based on the responses evoked by the one or more focused stimulation measurements.

In another aspect, a method is provided. The method comprises: performing a plurality of a same type of measurement on a recipient of an implantable medical device with a plurality of different degrees of focusing; obtaining responses evoked by each of the plurality of measurements; and collectively analyzing the responses evoked by each of the plurality of measurements relative to one another to determine one or more operational parameters of the implantable medical device.

In another aspect, one or more non-transitory computer readable storage media comprising instructions are provided. The instructions, when executed by a processor, cause the processor to: obtain at least one monopolar response of a recipient of an implantable medical device to at least one monopolar stimulation measurement performed on the recipient via the implantable medical device; obtain one or more focused responses of the recipient of the implantable medical device to one or more focused stimulation measurements performed on the recipient via the implantable medical device, wherein the at least one monopolar response and the one or more focused responses are a same type of response obtained from the recipient; and analyze the at least one monopolar response relative to the one or more focused responses; and based on the analyzing of the at least one monopolar response relative to the one or more focused responses, recommending one or more operational parameters for instantiation at the implantable medical device.

In another aspect, a computing device is provided. The computing device comprises: one or more network interface ports configured for communication with an implantable medical device system having an implantable component implantable within a recipient; a memory; and one or more processors configured to: initiate at least one monopolar stimulation measurement during which monopolar stimulation is delivered to the recipient via the implantable component; initiate one or more focused stimulation measurements during which focused stimulation is delivered to the recipient via the implantable component with varying degrees of focusing; obtain at least one monopolar response to the at least one monopolar stimulation measurement and one or more focused responses to the one or more focused stimulation measurements; and evaluate the one or more focused responses relative to the at least one monopolar response to determine one or more operational parameters for the implantable medical device system.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which certain embodiments presented herein may be implemented;

FIG. 1B is a side view of a recipient wearing a sound processing unit of the cochlear implant system of FIG. 1A;

FIG. 1C is a schematic view of components of the cochlear implant system of FIG. 1A;

FIGS. 1D is a block diagram of the cochlear implant system of FIG. 1A;

FIG. 2 is a high level flowchart of an example method, in accordance with certain embodiments presented herein;

FIG. 3 is a flowchart of another example method, in accordance with certain embodiments presented herein;

FIG. 4 is a schematic diagram illustrating differences in degree of focusing relative to stimulation channel bandwidth, in accordance with certain embodiments presented herein;

FIG. 5A is a schematic diagram illustrating a technique for setting a degree of focusing of a stimulation channel based on a critical bandwidth difference, in accordance with certain embodiments presented herein;

FIG. 5B is a schematic diagram illustrating a technique for setting a degree of focusing of a stimulation channel based on a critical change in slope, in accordance with certain embodiments presented herein;

FIG. 6 is a schematic diagram illustrating a technique for determining candidacy range for focused stimulation, in accordance with certain embodiments presented herein;

FIG. 7 is a diagram illustrating example measurements relative to granularity of performance measures, in accordance with certain embodiments presented herein;

FIG. 8 is a flowchart of a method for setting a degree of focusing across all stimulation channels of an electrode array based on a performance threshold, in accordance with certain embodiments presented herein.

FIG. 9 is a flowchart of a method for setting a degree of focusing for a subset of one or more stimulation channels based on a performance threshold, in accordance with certain embodiments presented herein.

FIG. 10 is a schematic diagram illustrating a fitting display screen, in accordance with certain embodiments presented herein;

FIG. 11 is a schematic diagram illustrating a vestibular nerve stimulator system with which certain embodiments presented herein may be implemented; and

FIG. 12 is a schematic block diagram of a computing device configured to, in accordance with certain embodiments, implement aspects of the techniques presented herein.

### DETAILED DESCRIPTION

Presented herein are techniques for setting operational parameters, such as stimulation parameters/settings, sound processing parameter/settings, etc., of an implantable medical device system based on a combination of at least one monopolar stimulation measurement and one or more multipolar (focused) stimulation measurements. In particular, the techniques presented herein perform at least one objective or behavioral measurement using monopolar stimulation, and the same objective or behavioral measurement using focused stimulation with one or more degrees of focusing. Hearing outcomes/responses evoked in response to each of the measurements are captured/recorded and evaluated relative to one another to set one or more operational parameters, such as the degree of focusing, of the implantable medical device or implantable medical device system.

Merely for ease of description, the techniques presented herein are primarily described with reference to a specific implantable medical device system, namely a cochlear implant system. However, it is to be appreciated that the techniques presented herein may also be implemented by other types of implantable medical devices or implantable medical device systems. For example, the techniques presented herein may be implemented by other auditory prosthesis systems that include one or more other types of auditory prostheses, such as middle ear auditory prostheses, bone conduction devices, direct acoustic stimulators, electro-acoustic prostheses, auditory brain stimulators, etc. The techniques presented herein may also be used with tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

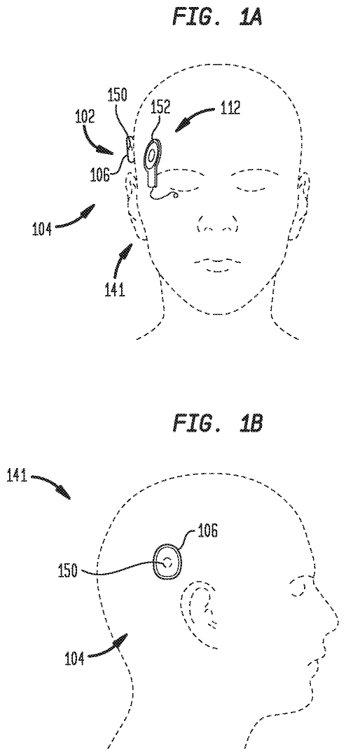

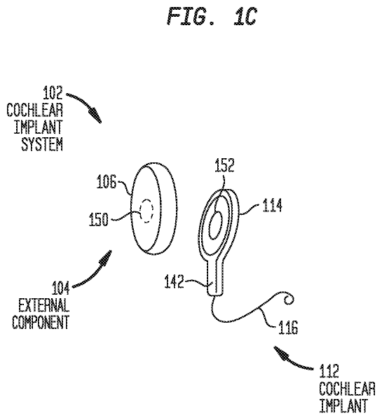

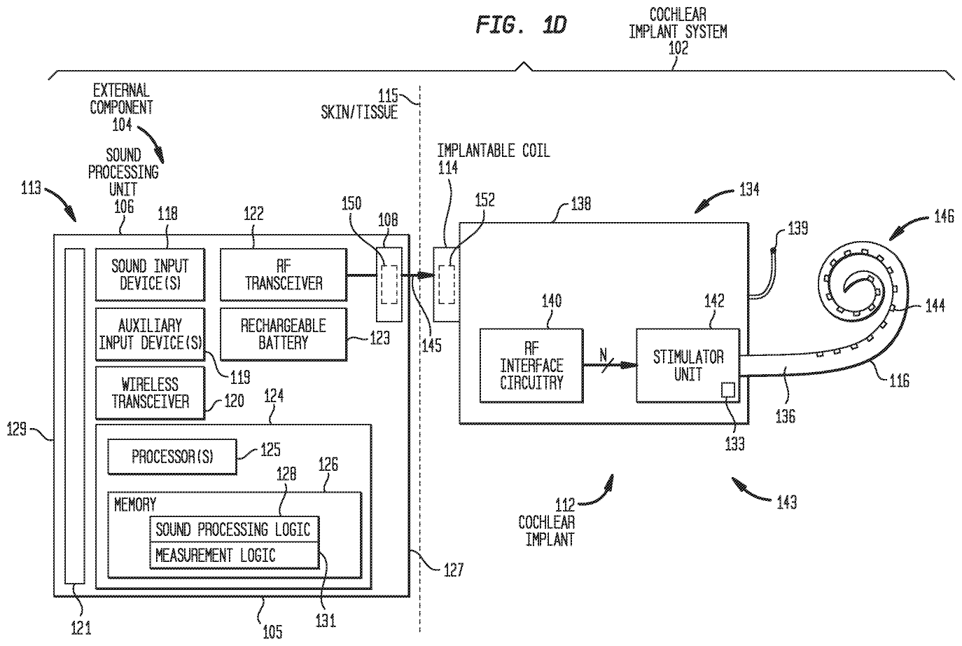

FIGS. 1A-1D are diagrams illustrating an example cochlear implant system 102 configured to implement certain embodiments of the techniques presented herein. The cochlear implant system 102 comprises an external component 104 and an implantable component 112. In the examples of FIGS. 1A-1D, the implantable component is sometimes referred to as a “cochlear implant.” FIG. 1A is a schematic diagram illustrating the implantable component 112 implanted in the head 141 of a recipient, while FIG. 1B is schematic drawing of the external component 104 worn on the head 141 of the recipient. FIG. 1C is another schematic view of the cochlear implant system 102, while FIG. 1D is a block diagram illustrating further details of the cochlear implant system 102. For ease of description, FIGS. 1A-1D will generally be described together.

As noted, cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the implantable component 112 includes an internal coil 114, a stimulator unit 142, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 105 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external coil 108 that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

FIGS. 1A-1D illustrate an arrangement in which the cochlear implant system 102 includes an external component. However, it is to be appreciated that embodiments of the present invention may be implemented in cochlear implant systems having alternative arrangements. For example, embodiments presented herein can be implemented by a totally implantable cochlear implant or other totally implantable medical device. A totally implantable medical device is a device in which all components of the device are configured to be implanted under skin/tissue of a recipient. Because all components are implantable, a totally implantable medical device operates, for at least a finite period of time, without the need of an external device/component. However, an external component can be used to, for example, charge the internal power source (battery) of the totally implantable medical device.

Returning to the specific example of FIGS. 1A-1D, FIG. 1D illustrates that the OTE sound processing unit 106 comprises one or more input devices 113 that are configured to receive input signals (e.g., sound or data signals). The one or more input devices 113 include one or more sound input devices 118 (e.g., microphones, audio input ports, telecoils, etc.), one or more auxiliary input devices 119 (e.g., audio ports, such as a Direct Audio Input (DAI), data ports, such as a Universal Serial Bus (USB) port, cable port, etc.), and a wireless transmitter/receiver (transceiver) 120. However, it is to be appreciated that one or more input devices 113 may include additional types of input devices and/or less input devices (e.g., the wireless transceiver 120 and/or one or more auxiliary input devices 119 could be omitted).

The OTE sound processing unit 106 also comprises the external coil 108, a charging coil 121, a closely-coupled transmitter/receiver (transceiver) 122, sometimes referred to as or radio-frequency (RF) transceiver 122, at least one rechargeable battery 123, and a processing module 124. The processing module 124 comprises one or more processors 125 and a memory device (memory) 126 that includes sound processing logic 128 and measurement logic 131. The memory device 126 may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors 125 are, for example, microprocessors or microcontrollers that execute instructions for the sound processing logic 128 and/or measurement logic 131 stored in memory device 126.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the transceiver 140 via a hermetic feedthrough (not shown in FIG. 1D). As shown in FIG. 1D, the stimulator can comprise measurement hardware 133, as described further below.

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 150 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link formed between the external coil 108 with the implantable coil 114. In certain examples, the closely-coupled wireless link is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the processing module 124. The processing module 124 is configured to convert received input signals (received at one or more of the input devices 113) into output signals for use in stimulating a first ear of a recipient (i.e., the processing module 124 is configured to perform sound processing on input signals received at the sound processing unit 106). Stated differently, the one or more processors 125 are configured to execute sound processing logic 128 in memory 126 to convert the received input signals into output signals 145 that represent electrical stimulation for delivery to the recipient.

As noted, FIG. 1D illustrates an embodiment in which the processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals 145) can be performed by a processor within the implantable component 112. That is, the implantable component 112, rather than the sound processing unit 106, could include a processing module that is similar to processing module 124 of FIG. 1D.

Returning to the specific example of FIG. 1D, the output signals 145 are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea via “stimulation channels,” where each stimulating channel comprises one or more of the electrodes 144. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As noted, a stimulation channel is a combination/set of the electrodes that are used simultaneously/collectively to deliver current signals to a recipient so as to elicit stimulation at a specific target location/place/region. The stimulation channels of cochlear implant system 102 (i.e., one or more electrodes), or other implantable medical device systems, can deliver stimulation to the recipient using different electrode configurations that each have different associated degrees of current spread (spread of excitation), referred to herein as “different degrees of focusing”. In general, the “focusing” or “current spread” associated with an electrode configuration refers to the width of the electric field created by the current signals delivered to the recipient via the one or more electrodes (e.g., the width along the frequency axis of an area of activated nerve cells in response to the delivered electrical stimulation).

Monopolar stimulation, for instance, is an electrode configuration where for a given stimulation channel the current is “sourced” via one of the electrodes 144, but the current is “sunk” by a remote electrode, such as the extra-cochlear electrode (ECE) 139 (FIG. 1D). Monopolar stimulation typically exhibits a large degree of current spread (i.e., wide stimulation pattern, referred to herein as a zero or no degree of focusing) and, accordingly, has a low spatial resolution. Other types of focused or “multipolar” electrode configurations, such as bipolar, tripolar, focused multi-polar (FMP), a.k.a. “phased-array” stimulation, etc. typically reduce the size of an excited neural population by “sourcing” the current via one or more of the electrodes 144, while also “sinking” the current via one or more other proximate electrodes, or in the case of focused multi-polar stimulation, by applying a pattern of positive and negative currents across multiple electrode contacts. Bipolar, tripolar, focused multi-polar and other types of electrode configurations that both source and sink current via intra-cochlear electrodes or reduce spread of excitation through positive and negative currents are generally and collectively referred to herein as “focused” stimulation and each exhibit different degrees of focusing. Focused stimulation typically exhibits a smaller degree of current spread (i.e., narrow stimulation pattern) when compared to monopolar stimulation and, accordingly, has a higher spatial resolution than monopolar stimulation. Likewise, other types of electrode configurations, such as double electrode mode, virtual channels, wide channels, defocused multi-polar, etc. typically increase the size of an excited neural population by “sourcing” the current via multiple neighboring intra-cochlear electrodes.

In general, it is desirable for a stimulation channel to stimulate only a narrow region of neurons such that the resulting neural responses from neighboring stimulation channels have minimal overlap. Accordingly, the ideal stimulation strategy in a cochlear implant would use focused stimulation channels to evoke perception of all sound signals at any given time. Such a strategy would also, ideally, enable each stimulation channel to stimulate a discrete tonotopic region of the cochlea to better mimic natural hearing and enable better perception of the details of the sound signals. Although focused stimulation generally improves hearing performance, this improved hearing performance comes at the cost of significant increased power consumption, added delays to the processing path, and increased complexity, etc., relative to the use of only monopolar stimulation. Additionally, not all recipients and/or not all stimulation channels benefit from the increased fidelity offered by focused stimulation.

Presented herein are techniques that use hearing outcomes obtained with both monopolar stimulation and focused stimulation to set one or more operational parameters of an implantable medical device. More specifically, in accordance with embodiments presented herein, a same objective or behavioral measurement is performed using monopolar stimulation and then using focused stimulation (e.g., with one or more degrees of focusing relative to monopolar stimulation). A measurement performed using monopolar stimulation is referred to herein as a “monopolar stimulation measurement” or “monopolar measurement,” while a measurement performed using focused stimulation is referred to herein as a “focused stimulation measurement” or “focused measurement.” The measurements can be objective measurements or behavioral/subjective measurements and may be conducted at one or more different sites of stimulation (e.g., a single stimulation channel, a group of stimulation channels, across the entire cochlea, etc.). The difference(s) in the responses/outcome between the monopolar stimulation measurement and the focused stimulation measurement are determined and then used to set one or more operational parameters, such as stimulation parameters, of the implantable medical device. In certain embodiments, the difference(s) in the outcome between the monopolar stimulation measurement and the focused stimulation measurement are used to determine the optimal degree of focusing to provide benefit for an individual at a given one or more stimulation channels or across the electrode array. In other embodiments, the difference(s) in the outcome between the monopolar stimulation measurement and the focused stimulation measurement are used to determine sound processing parameters, such as compression parameters, noise reduction parameters, sound processing strategy, etc.,

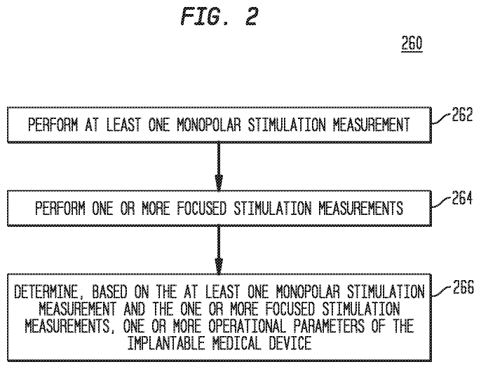

FIG. 2, below, is a flowchart illustrating an example method 260 in accordance with embodiments presented herein. Merely for ease of illustration, method 260 will be described with reference to cochlear implant system 102 of FIGS. 1A-1D. However, as noted elsewhere herein, it is to be appreciated that the techniques presented herein may be implemented in other types of implantable medical devices and/or implantable medical device systems.

Method 260 begins at 262 where at least one monopolar stimulation measurement is performed using one or more electrodes 144 of the cochlear implant 102 and the extra-cochlear electrode 139. That is, at least one objective or behavioral measurement is performed on the recipient by delivering monopolar stimulation to the recipient, where current is delivered/sourced via one or more of the electrodes 144, and the current is returned/sunk via the extra-cochlear electrode 139.

At 264, one or more focused stimulation measurements are performed using one or more of the electrodes 144 of the cochlear implant 102. That is, at least one objective or behavioral measurement (i.e., the same type of measurement as was performed at 262 to obtain the obtain the same type of response from the recipient) is performed on the recipient by delivering focused stimulation to the recipient where current is delivered/sourced via one or more electrodes 144, and the current is returned/sunk via one or more of the electrodes 144.

It is to be appreciated that, in certain embodiments, the one or more focused stimulation measurements may be performed prior to the at least one monopolar stimulation measurement. As such, the order of operations shown in FIG. 2 is merely illustrative. The at least one monopolar stimulation measurement and the one or more focused stimulation measurements can be performed, for example, using the measurement logic 131 (FIG. 1D), measurement hardware 133 (FIG. 1D), and/or, as described further below, can be initiated/controlled via an external computing device. In addition, it is to be appreciated that the monopolar stimulation measurements and the focused stimulation measurements are conducted either in the clinic or outside of the clinic (e.g., virtually, remotely, by the recipient using a computing device, etc.).

The at least one monopolar stimulation measurement and the one or more focused stimulation measurements can take a number of different forms and can comprise, for example, objective measurements (e.g., electrically evoked compound action potential (ECAP) measurements, Panoramic ECAP (PECAP) measurements, etc.) or behavioral measurements (e.g., speech tests, spectral ripple measurements, etc.). Further details of example objective and behavioral measurements are provided below. However, for a given stimulation site (one or more stimulation channels), the at least one monopolar stimulation measurement and the one or more focused stimulation measurements are the same type of objective or behavioral measurement obtaining/evoking the same type of response from the recipient. For example, if the at least one monopolar stimulation measurement is an ECAP measurement, then the one or more focused stimulation measurements also comprise ECAP measurements. In certain embodiments, the one or more focused stimulation measurements comprise a plurality of focused stimulation measurements that are performed with different degrees of focusing (e.g., different size electric fields).

Returning to the example of FIG. 2, the at least one monopolar stimulation measurement evokes at least one objective or behavioral “monopolar response,” while the one or more focused stimulation measurements evoke one or more objective or behavioral “focused responses.” At 266, the difference(s) in the responses/outcomes between the at least one monopolar stimulation measurement and the one or more focused stimulation measurements are determined and used to set (configure) one or more operational parameters, such as sound processing parameters/settings, stimulation parameter/settings, etc., of the cochlear implant system 102. That is, the at least one monopolar response (objective responses or behavioral response evoked by the at least one monopolar stimulation measurement) and the one or more focused responses (objective responses or behavioral responses evoked by the one or more focused stimulation measurements) are evaluated (e.g., compared) relative to one another. For example, as described further below, the difference(s) between the at least one monopolar response and the one or more focused responses is/are used to determine the optimal degree of focusing to provide benefit for the recipient at a given stimulation channel or across the electrode array 146.

A group/collection of at least one monopolar and one or more focused responses obtained from the same type objective or behavioral test, such as the responses obtained at 262 and 264, are sometimes referred to herein as a “monopolar-focused response set.” As such, certain examples presented herein refer to analysis of monopolar-focused response set in order to configure operational parameters of an implantable medical device or implantable medical device system.

The at least one monopolar response and the one or more focused responses can be collectively analyzed (e.g., evaluated relative to one another) in a number of different manners. In certain examples, the at least one monopolar response and the one or more focused responses can be collectively analyzed to determine whether or not a particular stimulation parameter, change in stimulation parameter, etc. provides a benefit to the recipient and/or determine whether the benefit of the parameter or change outweighs negative effects associated with the particular stimulation parameter, change in stimulation parameter, etc.

For example, the cochlear implant system 102 operates in accordance with a variety of pre-determined recipient-specific operational parameters/settings to convert processed audio data into one or more sets of stimulation signals. These operational parameters, sometimes referred to as the recipient's “map,” include, for example, electrode configuration or degree of focusing used to deliver stimulation signals at a given stimulation channel, channel-to-electrode mappings, stimulation/pulse rate, pulse timing (electrical pulse width and inter-pulse gap), mode of stimulation (polarity, reference electrode), compression law or compression settings, amplitude mappings, etc. In general, the operational parameters dictate how the processed audio signals are used for generation of sets of stimulation signals (current pulses) for delivery to the recipient via the various stimulation channels.

In cochlear implants, electrode configurations associated with higher degrees of focusing can improve spectral resolution compared to electrode configurations that deliver monopolar stimulation (or lesser degrees of focusing). However, as noted above, increasing degrees of focusing introduce increasingly greater complexity into the fitting (due to an additional parameter(s) that controls degree of focusing), but also require increasingly greater power consumption. Due to this focusing-power tradeoff, it is desirable not to increase focusing beyond the point that provides a benefit to hearing performance. The amount of benefit provided by focused stimulation may vary across different recipients and/or across different stimulation channels of a cochlear implant within a given recipient. Therefore, the degree of focusing should be specified for each recipient at one or more (ideally each) site of stimulation at a level which maximizes performance benefits while minimizing power needs.

Other operational parameters, such as channel selection, number of channels, or current steering may be modified along with degree of focusing to further improve hearing performance or reduce power consumption. Current methods for focusing optimization are clinically complex and time consuming.

As such, the techniques presented herein provide a method for setting the electrode configuration (e.g., degree of focused) for use in delivering stimulating signals via one or more stimulation channels, and/or other operational parameters, based on a collective analysis (e.g., relative evaluation) of monopolar stimulation responses and focused stimulation responses. In certain examples, this analysis includes a determination of whether focused stimulation, or increasing degrees of focusing, and/or other operational parameters provide a benefit for the recipient. That is, the techniques presented herein can determine the greatest possible benefit from focused stimulation (or a specific degree of focusing) without adding more focusing than is needed for the recipient at a given stimulation channel, group of stimulation channels, etc. If the focused stimulation (or a specific degree of focusing) does not improve hearing outcomes for the recipient, relative to monopolar, then greater focusing could lead to unnecessary power consumption. As described below, in some cases, the techniques presented herein provide additional power modeling to make decisions about focusing optimization. The thresholds for performance benefits and power consumption (battery life) may be set ahead of time by the clinician or the recipient. The threshold may be selected through clinical judgment, or may be selected through a balancing process to determine the relative weighting of performance and power.

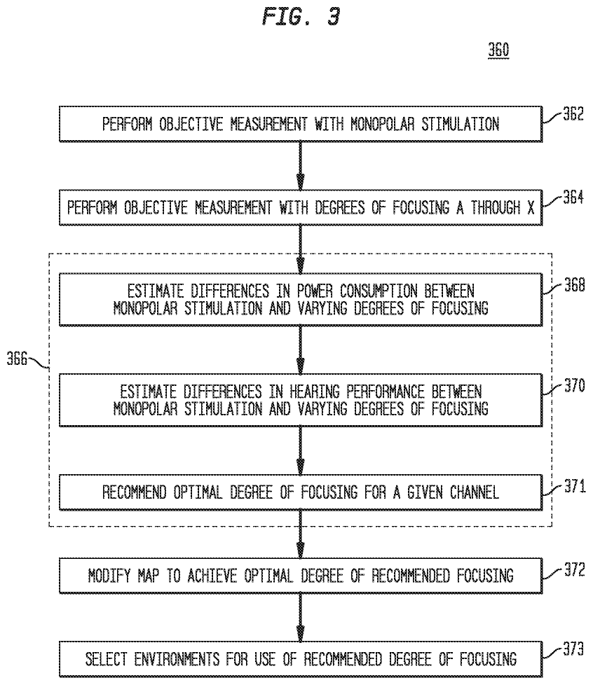

As noted above, the at least one monopolar stimulation measurement and the one or more focused stimulation measurements can be objective measurements or behavioral measurements. FIGS. 3-6 illustrate an example use of objective measurements to set operational parameters. Again, merely for ease of illustration, the examples of FIGS. 3-6 will be described with reference to cochlear implant system 102 of FIGS. 1A-1D.

Referring first to FIG. 3, shown is a flowchart of an example method 360 for use of objective metrics of hearing performance and modification of map parameters, taking into account the potential tradeoff between power consumption and hearing performance. For ease of reference, method 360 is described as being performed at a single stimulation channel. It is to be appreciated that method 360 could, in practice, be performed for multiple stimulation channels (e.g., sequentially, simultaneously across multiple stimulation channels, etc.)

Method 360 begins at 362 where at least one objective monopolar stimulation measurement is performed using one or more electrodes 144 of the cochlear implant 102 and the extra-cochlear electrode 139 (e.g., perform objective measurement is performed on the recipient by delivering monopolar stimulation to the recipient). At 364, a plurality of objective focused stimulation measurements are performed using one or more of the electrodes 144 of the cochlear implant 102, where the objective focused stimulation measurements are performed with different degrees of focusing, referred to as degrees of focusing a through n (e.g., the same type of objective measurement as was performed at 362, but by delivering focused stimulation to the recipient). The use of “different degrees of focusing” means that different ones of the plurality of objective focused stimulation measurements are performed using electrode configurations that result in different amounts of focusing (e.g., different sized electric fields). Reference to “a through n” means there are “n” different degrees of focusing used to perform the plurality of objective focused stimulation measurements.

At the end of 364, the system has obtained/captured an objective monopolar response (response evoked by the monopolar stimulation) and a plurality of objective focused responses, with different degrees of focusing (e.g., a monopolar-focused response set including one monopolar response and a plurality of focused responses).

At 366, the difference(s) in the responses/outcomes between monopolar response and the plurality of focused stimulation measurements are determined and used to set (configure) one or more operational parameters of the cochlear implant system 102. Shown in FIG. 3 is a specific example of the operations at 366 for use of the difference(s) in the responses/outcomes between monopolar response and the plurality of focused stimulation measurements to set one or more operational parameters of the cochlear implant system 102. More specifically, the operations of 366 begin at 368 where an estimate in the differences in power consumption between the monopolar stimulation and each of the varying degrees of focused stimulation is determined. At 370, an estimate in the differences in hearing performance between the monopolar stimulation and each of the varying degrees of focused stimulation is determined. At 371, the differences in power consumption and the differences in hearing performance are analyzed to recommend a degree of focusing for the stimulation channel.

At 372, the recipient's map is modified to achieve the recommended degree of focusing at the stimulation channel. In certain examples, method 360 ends after 372 and the recommended degree of focusing at the stimulation channel is used regardless of the sound input. However, FIG. 3 illustrates a specific example in which, at 373, in which the recommended degree of focusing at the stimulation channel is only used in certain sound environments. More specifically, the sound processing logic 128 (FIG. 1D) is configured to evaluate/analyze the input sound signals and determine the sound class of the sound signals. That is, the sound processing logic 128 is configured to use the received sound signals to “classify” the ambient sound environment and/or the sound signals into one or more sound categories (i.e., determine the input signal type). The sound classes/categories may include, but are not limited to, “Speech,” “Noise,” “Speech+Noise,” “Music,” and “Quiet.” At 373 of FIG. 3, a determination is made to use the recommended degree of focusing at the stimulation channel only in certain sound classes. This decision may be based, for example, based on recipient preferences or inputs, automated settings, etc.

In summary, FIG. 3 illustrates an example in which objective metrics are obtained using monopolar stimulation as well as focused stimulation with varying degrees of focusing and the differences in these metrics are used to predict differences in performance outcomes for a given degree of focusing. In certain embodiments, a tuning curve representing the objective metric as a function of degree of focusing can be generated and used to set the degree of focusing. If the curve reaches a critical point at which the response stops changing, then further increases in focusing are not expected to provide benefit for the recipient.

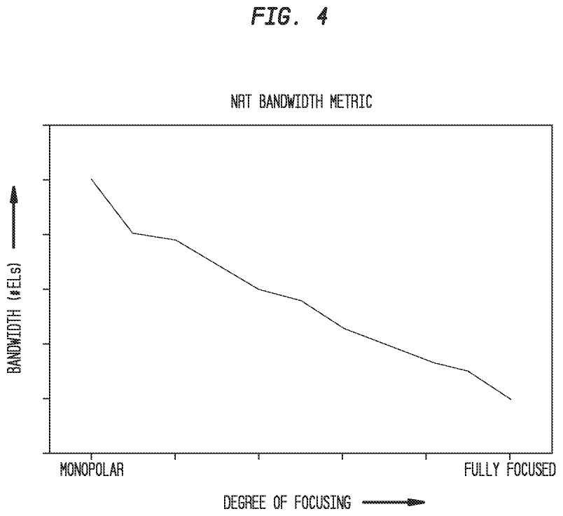

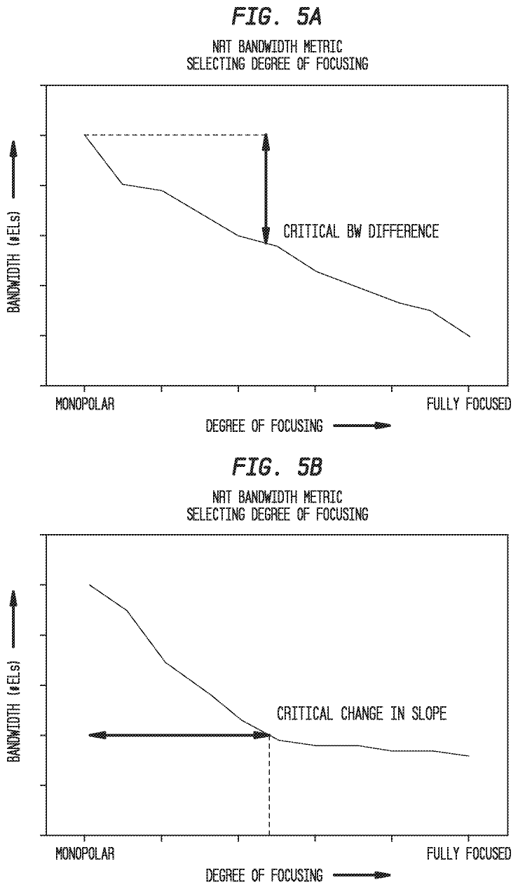

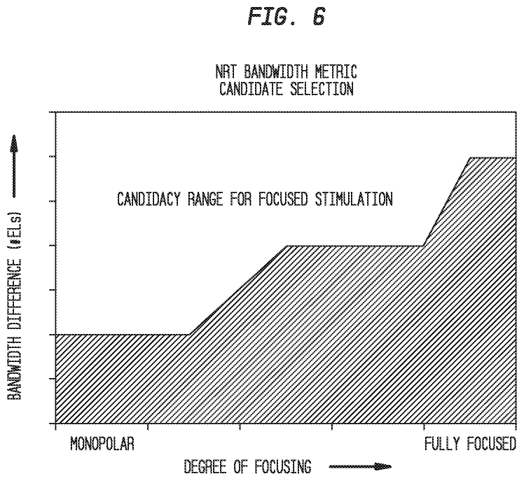

FIGS. 4, 5A, 5B, and 6 illustrate example tuning curves generated from electrically evoked compound action potentials (ECAP), also referred to as neural response telemetry (NRT) responses. FIGS. 4, 5A, and 5B all illustrate examples in which focused stimulation provides sharper tuning compared to monopolar stimulation, and that increased focusing results in greater sharpness (reduced bandwidth/reduced electrical field width). However, as noted above, a greater amount of focusing may not be appropriate in all situations and, as such, there is a need to determine an optimal or recommend amount of focusing at a given stimulation channel.

In FIG. 5A, the optimal degree of focusing is selected for a given stimulation channel based on an established critical bandwidth difference. That is, in this example, the optimal degree of focusing is set at which a critical bandwidth difference is achieved. It is to be appreciated that the critical bandwidth is just one example of a “delta performance” with an objective metric, which can be set using clinical judgment or through the power/performance weighting exercise, as described below.

In FIG. 5B, an optimal degree of focusing is selected based on an observed change in the amount of improvement along the function (i.e., decrease in slope). It is to be appreciated that FIG. 5B illustrates a slightly different delta performance, as compared to FIG. 5A, because FIG. 5B involves measurement of the full function to find the slope, whereas with a critical bandwidth difference the process stops upon reaching the defined delta.

FIG. 6 illustrates an example curve for candidate selection. In this example, the recipient must demonstrate a difference in bandwidth beyond the criterion improvement at a given degree of focusing to be considered a good candidate for focused stimulation. In other words, for greater degrees of focusing to be recommended, the greater focusing should result in greater differences in bandwidth (predicting larger performance improvements) due to the tradeoff between degree of focusing and power consumption.

The examples of FIGS. 4, 5A, 5B, and 6 are merely illustrative of example determinations performed for a single stimulation channel. It is to be appreciated that similar determinations may be made at one or more stimulation channels along the electrode array. It is also to be appreciated that the use of ECAP/NRT is merely illustrative and that similar determinations may be made based on other objective measurements, such as Panoramic ECAP, neural envelope tracking, higher evoked potentials measured from the brainstem and auditory cortex, measurements of the electrical properties of the recipient's tissue, and/or the electrode array, interaction-based measurements of neural excitation, pupillometry, etc. It is to be appreciated that this list of objective measurements is for exemplary purposes and is not exhaustive.

As noted, in certain embodiments presented herein, monopolar behavioral measurements and focused behavioral measurements may also be used to set operational parameters. For example, in certain embodiments, the behavioral measurements can comprise speech tests performed using monopolar stimulation and focused stimulation, potentially with different degrees of focusing.

In a speech test, a speech stimulus or speech stimulation (monopolar in the first phase and focused stimulation in the second and subsequent phases) representing various “speech components,” such as phonemes, words, sentences, etc., is presented to the recipient and the recipient is asked to identify what she heard (e.g., respond verbally, respond using an electronic device, etc.). The speech components can be presented with or without background noise. Data is obtained regarding how well the recipient recognized the speech components with each type of stimulation (e.g., calculate the percent of correct responses).

Speech tests may further include or comprise a speech reception threshold (dB) component in which a speech stimulus (e.g., representing sentences, words, phonemes, digits, etc.) is presented to the recipient in the presence of background noise. The speech and/or the background noise is then adjusted to determine the threshold at which the user can just identify the stimulus.

In certain embodiments, the behavioral measurements can comprise psychophysical tests performed using monopolar stimulation and focused stimulation, potentially with different degrees of focusing. For example, the psychophysical tests can comprise spectral ripple tests in which a stimulus is presented with a noise or multi-tone carrier and spectral modulations. The depth or density of the modulations are adjusted to determine the threshold at which the recipient can just detect a difference across stimuli. In other examples, the psychophysical tests can comprise electrode discrimination tests to determine the smallest difference in a number of electrodes that a recipient can detect.

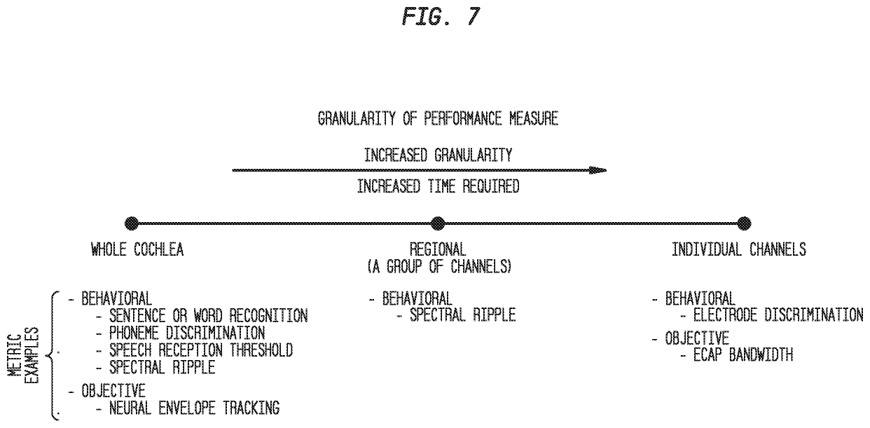

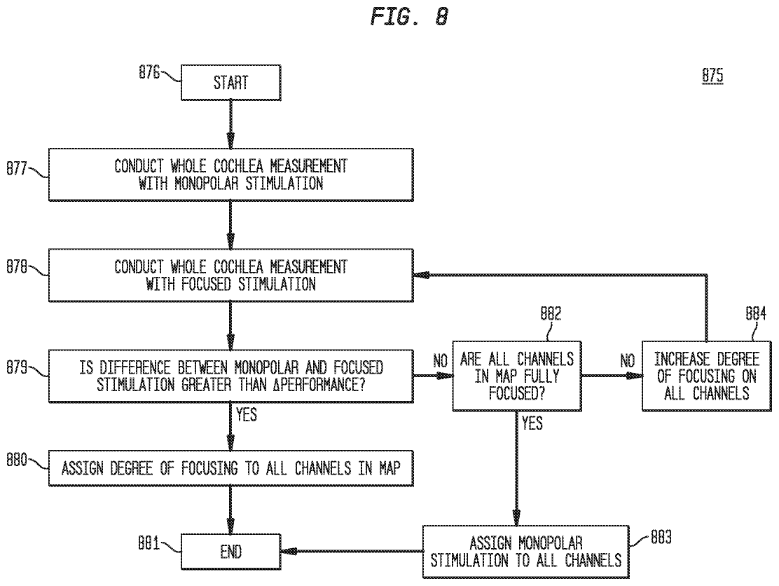

In accordance with embodiments presented herein, both behavioral and objective measures may be conducted with different levels of granularity (e.g., different number of stimulation channels). Entire cochlea measures that are conducted with a full map will generally be less time consuming that performing measures in a particular cochlear region or for individual channels. FIG. 7 provides examples of behavioral and objective performance measures that may be used with different levels of granularity.

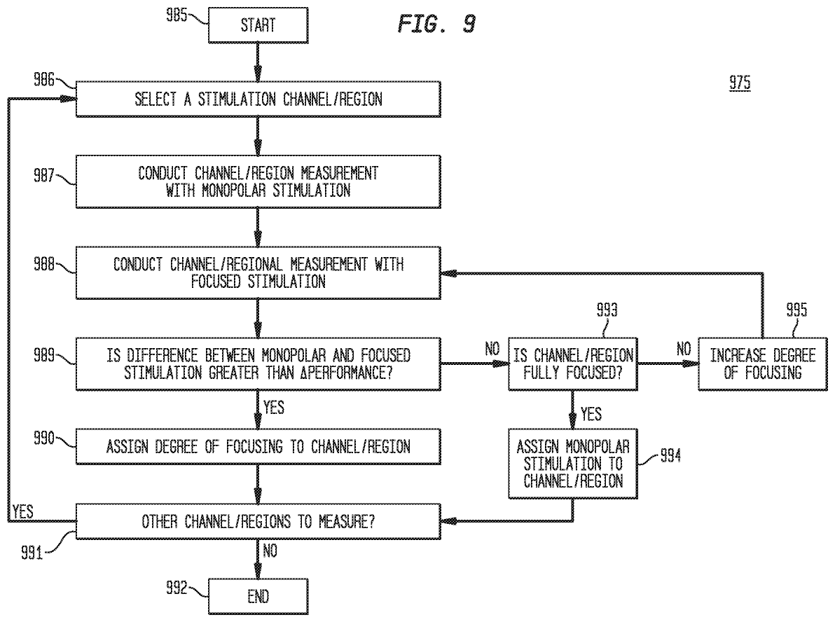

In accordance with embodiments presented herein, setting the degree of focusing (or other operational parameters) relies upon outcomes/responses evoked in response to monopolar stimulation and in response to different degrees of focused stimulation. Shown in FIGS. 8 and 9, are methods for determining a recommended degree of focusing based upon a clinically relevant pre-determined threshold for the difference in benefit to recipient between monopolar and focused stimulation, where the threshold is labeled “ΔPerformance.” That is, ΔPerformance represents the minimum benefit that a particular degree of focusing needs to provide over monopolar stimulation for the particular degree of focusing to be used within the recipient's map.

In the examples of FIGS. 8 and 9, the selected degree of focusing is driven by the benefits provided by focusing to the recipient (e.g., in terms of objective measures, speech understanding, etc.). The measurements start with the lowest amount of focusing to avoid providing more focusing than is necessary to help account for power consumption. However, no changes are made based on power outcomes.

Referring first to FIG. 8, a method 875 is shown performed across an entire electrode array. Method 875 starts at 876 and, at 877, a monopolar stimulation measurement is performed across all stimulation channels of the electrode array. At 878, a focused stimulation measurement is performed across all stimulation channels of the electrode array. At 879, a determination is made as to whether or not the difference in performance between the monopolar stimulation measurement and the focused stimulation measurement is greater than ΔPerformance (e.g., is the performance difference greater than the clinically relevant pre-determined threshold). If the difference in performance is greater than ΔPerformance, then the method 875 continues to 880 where the degree of focusing used to perform the most recent focused stimulation measurement is assigned to all of the stimulation channels for use as part of the recipient's map. The method then ends at 881.

Returning to 879, if it is determined that the difference in performance is not greater than ΔPerformance, then the method continues to 882 where a determination is made as to whether or not all stimulation channels are fully focused (i.e., has the maximum amount of available focusing been used to perform the most recent focused stimulation measurement). If all stimulation channels are fully focused, then method 875 continues to 883 where monopolar stimulation is assigned to all channels. In other words, if all stimulation channels are fully focused and the threshold ΔPerformance has not yet been exceeded, then it is determined that focused stimulation is not benefiting the recipient and that monopolar stimulation should be used for all stimulation channels. The method then ends at 881.

Returning to 882, if it is determined that all stimulation channels are not fully focused (i.e., the maximum amount of available focusing has not been used to perform the most recent focus measurement), then method 875 continues to 884. At 884, the degree of focusing is increased for all stimulation channels, and the method then returns to 878 where the focused stimulation measurement is performed using the increased degree of focusing. The operations of 878, 879, 882, and 884 are repeated iteratively until the difference in performance is greater than ΔPerformance (at 880) or all stimulation channels are fully focused (at 883), after which the method 875 subsequently ends at 881.

Referring next to FIG. 9, a method 975 is shown performed at one or more individual stimulation channels/regions in a sequential manner. More specifically, method 975 starts at 985 and, at 986, a stimulation channel/region is selected. At 987, a monopolar stimulation measurement is performed at the selected stimulation channel/region. At 988, a focused stimulation measurement is performed at the selected stimulation channel/region. At 979, a determination is made as to whether or not the difference in performance between the monopolar stimulation measurement and the focused stimulation measurement is greater than ΔPerformance (e.g., the clinically relevant pre-determined threshold for a target difference). If the difference in performance is greater than ΔPerformance, then the method 975 continues to 990 where the degree of focusing used to perform the focused stimulation measurement is assigned to the selected stimulation channel/region within the recipient's map.

At 991, a determination is made as to whether or not measurements should be performed at additional stimulation channels/regions. If no additional measurements are to be performed, then the method 975 ends at 992. However, if additional measurements should be performed at additional stimulation channels/regions, then the method 975 returns to 986 where another stimulation channel/region is selected and the above steps are repeated.

Returning to 989, if it is determined (during any iteration of the method) that the difference in performance is not greater than ΔPerformance, then the method continues to 993 where a determination is made as to whether or the selected stimulation channel/region is fully focused (i.e., has the maximum amount of available focusing been used to perform the most recent focused stimulation measurement). If the selected stimulation channel/region is fully focused, then method 975 continues to 994 where monopolar stimulation is assigned to the selected stimulation channel/region. In other words, if the selected stimulation channel/region is fully focused and the threshold ΔPerformance has not yet been exceeded, then it is determined that focused stimulation is not benefiting the recipient at the selected stimulation channel/region and that monopolar stimulation should be used for the selected stimulation channel/region. The method then continues to 991, as described above.

Returning to 993, if it is determined that the selected stimulation channel/region is not fully focused (i.e., the maximum amount of available focusing has not been used to perform the most recent focus measurement), then method 975 continues to 995. At 995, the degree of focusing is increased for the selected stimulation channel/region, and the method then returns to 988 where the focused stimulation measurement is performed using the increased degree of focusing (at the selected stimulation channel/region). The operations of 988, 989, 993, and 995 are repeated iteratively until the difference in performance is greater than ΔPerformance (at 989) or all stimulation channels are fully focused (at 993), after which the method 975 subsequently continues to 991, as described above. Method 975 ends at 992 when each of the desired/selected stimulation channel/regions have been analyzed.

As noted above, FIGS. 8 and 9 generally illustrate methods in which the determination of which degree of focusing to use at one or more stimulation channels is based on whether a particular degree of focusing benefits the recipient at the one or more stimulation channels (e.g., degree of focusing is driven by the benefits provided by the focusing). However, it is to be appreciated that the determination of which degree of focusing to use at one or more stimulation channels could also or alternatively be based on additional information. In certain embodiments, the determination of which degree of focusing to use at one or more stimulation channels is based on based on whether the focusing benefits the recipient at the one or more stimulation channels, as well as power consumption data. In such embodiments, of the methods of 875 and 975 could be modified to include another determination loop that evaluates the power consumed by a given degree of focusing. As such, even though an increased degree of focusing may benefit the recipient, if such degree of focusing consumes too much power, the system could set the degree of focusing to a lower degree (e.g., tradeoff between power and performance benefits).

In certain embodiments, the ΔPerformance threshold could be generated based on a user-driven input weighting accounting for factors such as performance, power, etc. For example, the recipient, clinician or other user could provide input on what factors are most important to the specific recipient, such as hearing performance (e.g., how well the recipient understands speech, music, or other signals), power (e.g., how long the battery is likely to last), etc.

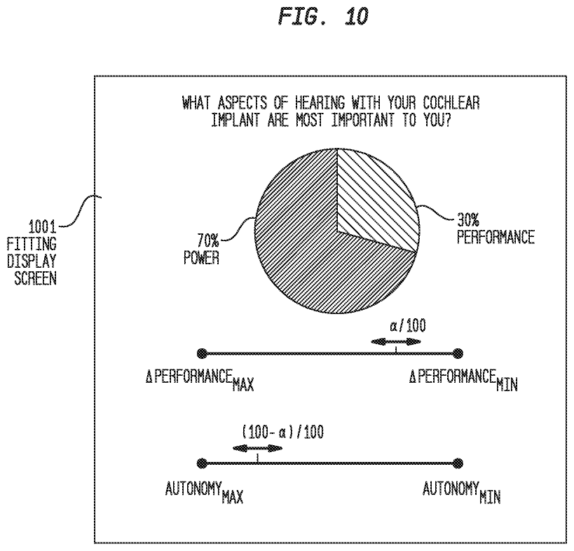

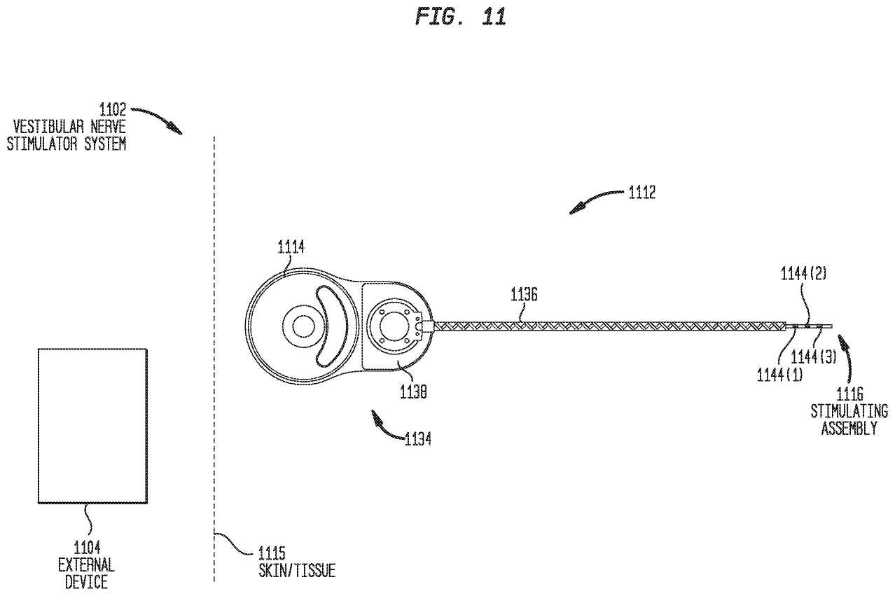

FIG. 10 illustrates a fitting display screen 1001 that, in accordance with certain specific embodiments, instructs the recipient to move a slider to select a balance between Performance % (α) and Power % (100−α), which always will add up to 100%. This balance will define the goals of the fitting and the performance/power tradeoff In particular, the % Performance selected, α, will be used to scale the ΔPerformance_{Focused-Monopolar} (i.e., the critical performance difference) between a minimum and a maximum value (FIG. 10). Similarly, (100−α), the % Power, will be used to scale the target number of battery hours between Autonomyₘₐₓ and Autonomyₘᵢₙ (FIG. 10). Provided below are several illustrative examples of the embodiments of FIG. 10.

### EXAMPLE 1

If α=100% Performance,
ΔPerformance=ΔPerformanceₘₐₓ
Autonomy=Autonomyₘᵢₙ

### EXAMPLE 2

If α=0, or 100% Power
ΔPerformance=ΔPerformanceₘᵢₙ
Autonomy=Autonomyₘₐₓ

### EXAMPLE 3

If 0<α<100, then the ΔPerformance required to switch from monopolar to focused, and the autonomy threshold are determined as follows:

$$∘ Δ Performance = Δ {Performance}_{min} + \frac{α}{100} ( Δ {Performance}_{max} - Δ {Performance}_{min} )$$

$$∘ Autonomy = {Autonomy}_{min} + \frac{( 100 - α )}{100} ( {Autonomy}_{max} - {Autonomy}_{min} )$$

It is possible that the targets for ΔPerformance and battery autonomy will not both be met as part of the fitting session. In that case, the available battery autonomy and ΔPerformance will be presented to the clinician/recipient to determine if the outcome is acceptable. For example, if the system is at a Power % equal to 70%, the target is Amin+70% of the difference between Amax and Amin.

As noted, FIG. 10 illustrates one example technique for performance/power weighting as a user-driven input to selecting the degree of focusing. When using performance/power weighting as an input, the process includes thresholds for both ΔPerformance and battery autonomy.

Embodiments have primarily been described herein with referring to setting or determining a degree of focusing for use in delivering stimulation signals one or more stimulation channels of an implantable medical device. However, as noted elsewhere herein, the techniques presented herein may be used to set or determine a number of operational parameters of an implantable medical device. For example, the techniques presented herein may be used to perform channel selection (e.g., determine a set of stimulation channels for use in delivering stimulation signals to the recipient of the implantable medical device), to configure current steering (e.g., determine current steering parameters for use in delivering stimulation signals at one or more locations between stimulation contacts of the implantable medical device), etc. In certain embodiments, the techniques presented herein may be used to determine combinations of degree of focusing, channel selection, and/or current steering.

Embodiments presented herein have been primarily described with reference to an example auditory prosthesis system, namely a cochlear implant system. However, as noted above, it is to be appreciated that the techniques presented herein may be implemented by a variety of other types of implantable medical devices (or systems that include other types of implantable medical devices) that provide a wide range of therapeutic benefits to recipients, patients, or other users. For example, the techniques presented herein may be implemented by other auditory prostheses, such as acoustic hearing aids, middle ear auditory prostheses, bone conduction devices, direct acoustic stimulators, electro-acoustic prostheses, other electrically simulating auditory prostheses (e.g., auditory brain stimulators), etc. The techniques presented herein may also be implemented by tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

FIG. 11 illustrates an example vestibular stimulator system 1102, with which embodiments presented herein can be implemented. In particular, one or more operational parameters/settings of the vestibular stimulator system 1102 may be set/determined based on a relative analysis of different degrees of focusing, such as a relative analysis of monopolar stimulation measurements and one or more focused stimulation measurements.

As shown, the vestibular stimulator system 1102 comprises an implantable component (vestibular stimulator) 1112 and an external device/component 1104 (e.g., external processing device, battery charger, remote control, etc.). The vestibular stimulator 1112 comprises an implant body (main module) 1134, a lead region 1136, and a stimulating assembly 1116, all configured to be implanted under the skin/tissue (tissue) 1115 of the recipient. The implant body 1134 generally comprises a hermetically-sealed housing 1138 in which RF interface circuitry, one or more rechargeable batteries, one or more processors, and a stimulator unit are disposed. The implant body 134 also includes an internal/implantable coil 1114 that is generally external to the housing 1138, but which is connected to the transceiver via a hermetic feedthrough (not shown).

The stimulating assembly 1116 comprises a plurality of electrodes 1144 disposed in a carrier member (e.g., a flexible silicone body). In this specific example, the stimulating assembly 1116 comprises three (3) stimulation electrodes, referred to as stimulation electrodes 1144(1), 1144(2), and 1144(3). The stimulation electrodes 1144(1), 1144(2), and 1144(3) function as an electrical interface for delivery of electrical stimulation signals to the recipient's vestibular system.

The stimulating assembly 1116 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

The methods of, for example, FIGS. 2, 3, 8, and/or 9, as well as other methods presented herein, could be implemented with, or partially by, the vestibular stimulator system 1102 to set one or more operational parameters of the vestibular stimulator system.

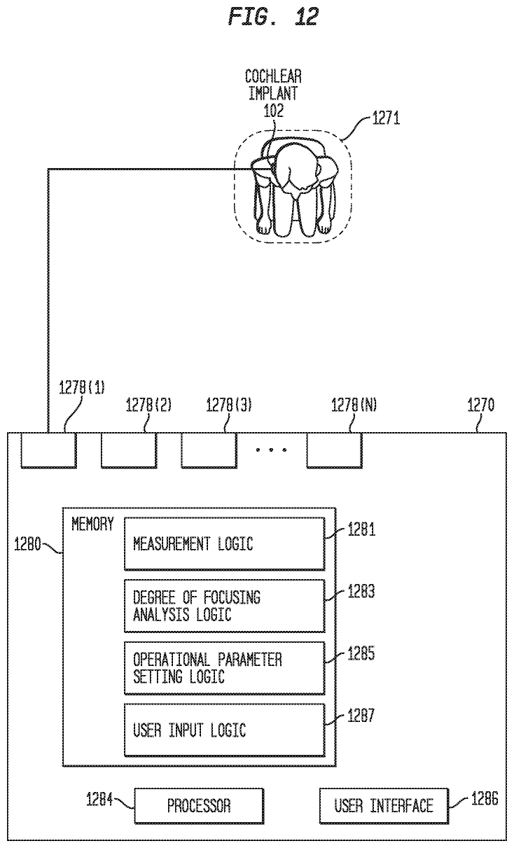

Aspects of the techniques presented herein may, in certain embodiments, be implemented at an external computing device in communication with an implantable medical device system. FIG. 12 is a block diagram of one such example computing device 1270 that, for ease of illustration, will be described with reference to cochlear implant system 102.

The computing device 1270 may comprise, for example, a fitting system, computer (e.g., laptop, desktop, tablet, etc.), a mobile device (e.g., mobile phone), etc., configured to communicate with the cochlear implant system 102. In the example of FIG. 12, the computing device 1270 comprises a plurality of interfaces/ports 1278(1)-1278(N), a memory 1280, a processor 1284, and a user interface 1286. The interfaces 1278(1)-1278(N) may comprise, for example, any combination of network ports (e.g., Ethernet ports), wireless network interfaces, Universal Serial Bus (USB) ports, Institute of Electrical and Electronics Engineers (IEEE) 1394 interfaces, PS/2 ports, etc. In the example of FIG. 12, interface 1278(1) is connected to the cochlear implant system 102 having components implanted in a recipient 1271. Interface 1278(1) may be directly connected to the cochlear implant system 102 or connected to an external device that is communication with the cochlear implant system. Interface 1278(1) may be configured to communicate with cochlear implant system 102 via a wired or wireless connection (e.g., telemetry, Bluetooth, etc.).

The user interface 1286 includes one or more output devices, such as a liquid crystal display (LCD) and a speaker, for presentation of visual or audible information to a clinician, audiologist, or other user. The user interface 1286 may also comprise one or more input devices that include, for example, a keypad, keyboard, mouse, touchscreen, etc.

The memory 1280 comprises measurement logic 1281 that can be executed to perform monopolar and focused stimulation measurements via the cochlear implant system 102, as described above. As shown, memory 1280 also comprises degree of focusing analysis logic 1283 that can be executed to analyze the monopolar and focused stimulation measurements and recommend a degree of focusing and/or other operational parameters for one or more stimulation channels of the cochlear implant system 102. Also shown in FIG. 12 is stimulation parameter setting logic 1285 that can be executed to set/configure the cochlear implant system 102 with the recommended degree of focusing and/or other operational parameters for one or more stimulation channels of the cochlear implant system 102 (e.g., send the operational parameters to the cochlear implant system 102 for instantiation as part of the recipient's map). Finally, shown in FIG. 12 is user input logic 1287 that can be executed to receive user-driven data for use in recommending a degree of focusing and/or other operational parameters for one or more stimulation channels of the cochlear implant system 102.

Memory 1280 may comprise read only memory (ROM), random access memory (RAM), magnetic disk storage media devices, optical storage media devices, flash memory devices, electrical, optical, or other physical/tangible memory storage devices. The processor 1284 is, for example, a microprocessor or microcontroller that executes instructions for the logic 1281, 1283, 1285, and 1287 stored in the memory 1280. Thus, in general, the memory 1280 may comprise one or more tangible (non-transitory) computer readable storage media (e.g., a memory device) encoded with software comprising computer executable instructions and when the software is executed (by the processor 1284) it is operable to perform the techniques described herein.

It is to be appreciated that the embodiments presented herein are not mutually exclusive and that the various embodiments may be combined with another in any of a number of different manners.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A method, comprising:
performing at least one monopolar stimulation measurement on a recipient of an implantable medical device;
performing one or more focused stimulation measurements on the recipient of the implantable medical device, wherein the at least one monopolar stimulation measurement and the one or more focused stimulation measurements obtain the same type of response from the recipient; and
determining one or more operational parameters of the implantable medical device based on the responses evoked by at least one monopolar stimulation measurement and based on the responses evoked by the one or more focused stimulation measurements.

2. The method of claim 1, wherein performing at least one monopolar stimulation measurement on the recipient comprises:
performing at least one objective monopolar stimulation measurement on the recipient, and
wherein performing one or more focused stimulation measurements on the recipient comprises:
performing one or more objective focused stimulation measurements on the recipient.

3. The method of claim 2, wherein the at least one objective monopolar stimulation measurement and the one or more focused stimulation measurements are electrically evoked compound action potential (ECAP) measurements.

4. The method of claim 1, wherein performing at least one monopolar stimulation measurement on the recipient comprises:
performing at least one behavioral monopolar stimulation measurement on the recipient, and
wherein performing one or more focused stimulation measurements on the recipient comprises:
performing one or more behavioral focused stimulation measurements on the recipient.

5. The method of claim 4, wherein the at least one behavioral monopolar stimulation measurement and the one or more behavioral stimulation measurements are speech tests.

6. The method of claim 4, wherein the at least one behavioral monopolar stimulation measurement and the one or more behavioral stimulation measurements are psychophysical tests.

7. The method of claim 1, wherein performing one or more focused stimulation measurements on the recipient comprises:
performing a plurality of focused stimulation measurements on the recipient with different degrees of focusing.

8. The method of claim 1, wherein determining one or more operational parameters of the implantable medical device based on the responses evoked by at least one monopolar stimulation measurement and based on the responses evoked by the one or more focused stimulation measurements, comprises:
estimating one or more differences in hearing performance between the at least one monopolar stimulation measurement and the one or more focused stimulation measurements; and
setting the one or more operational parameters based on the one or more differences in hearing performance between the at least one monopolar stimulation measurement and the one or more focused stimulation measurements.

9. The method of claim 1, wherein determining one or more operational parameters of the implantable medical device based on the responses evoked by at least one monopolar stimulation measurement and based on the responses evoked by the one or more focused stimulation measurements, comprises:
estimating one or more differences in hearing performance between the at least one monopolar stimulation measurement and the one or more focused stimulation measurements;
estimating one or more differences in power consumption between the at least one monopolar stimulation measurement and the one or more focused stimulation measurements; and
setting the one or more operational parameters based on the one or more differences in hearing performance and the one or more differences in power consumption between the at least one monopolar stimulation measurement and the one or more focused stimulation measurements.

10. The method of claim 1, wherein determining one or more operational parameters of the implantable medical device based on the responses evoked by at least one monopolar stimulation measurement and based on the responses evoked by the one or more focused stimulation measurements, comprises:
determining a degree of focusing for use in delivering stimulation signals at one or more stimulation channels of the implantable medical device.

11. The method of claim 1, wherein determining one or more operational parameters of the implantable medical device based on the responses evoked by at least one monopolar stimulation measurement and based on the responses evoked by the one or more focused stimulation measurements, comprises:
determining a set of stimulation channels for use in delivering stimulation signals to the recipient of the implantable medical device.

12. The method of claim 1, wherein determining one or more operational parameters of the implantable medical device based on the responses evoked by at least one monopolar stimulation measurement and based on the responses evoked by the one or more focused stimulation measurements, comprises:
determining current steering parameters for use in delivering stimulation signals to one or more locations between stimulation contacts of the implantable medical device.

13. A method, comprising:
performing a plurality of a same type of measurement on a recipient of an implantable medical device with a plurality of different degrees of focusing;
obtaining responses evoked by each of the plurality of measurements; and
collectively analyzing the responses evoked by each of the plurality of measurements relative to one another to determine one or more operational parameters of the implantable medical device.

14. The method of claim 13, wherein performing a plurality of the same type of measurement on the recipient of the implantable medical device with the plurality of different degrees of focusing comprises:
performing at least one monopolar stimulation measurement on the recipient; and
performing one or more focused stimulation measurements on the recipient.

15. The method of claim 13, wherein performing a plurality of the same type of measurement on the recipient of the implantable medical device with the plurality of different degrees of focusing comprises:
performing at least one monopolar stimulation measurement on the recipient; and
performing a plurality of focused stimulation measurements on the recipient, wherein each of the focused stimulation measurements utilize different degrees of focusing.

16. The method of claim 13, wherein performing a plurality of the same type of measurement on the recipient of the implantable medical device with the plurality of different degrees of focusing comprises:
performing a plurality of the same type of objective measurement on the recipient.

17. (canceled)

18. The method of claim 13, wherein performing a plurality of the same type of measurement on the recipient of the implantable medical device with the plurality of different degrees of focusing comprises:
performing a plurality of the same type of behavioral measurement on the recipient.

19. (canceled)

20. The method of claim 13, wherein collectively analyzing the responses evoked by each of the plurality of measurements relative to one another to determine one or more operational parameters of the implantable medical device comprises:
collectively analyzing the responses evoked by each of the plurality of measurements relative to one another to determine a degree of focusing for use in delivering stimulation signals at one or more stimulation channels of the implantable medical device.

21. The method of claim 13, wherein collectively analyzing the responses evoked by each of the plurality of measurements relative to one another to determine one or more operational parameters of the implantable medical device comprises:
collectively analyzing the responses evoked by each of the plurality of measurements relative to one another to determine a set of stimulation channels for use in delivering stimulation signals to the recipient of the implantable medical device.

22. The method of claim 13, wherein collectively analyzing the responses evoked by each of the plurality of measurements relative to one another to determine one or more operational parameters of the implantable medical device comprises:
collectively analyzing the responses evoked by each of the plurality of measurements relative to one another to determine current steering parameters for use in delivering stimulation signals to one or more locations between stimulation contacts of the implantable medical device.

23. (canceled)

24. (canceled)

25. One or more non-transitory computer readable storage media comprising instructions that, when executed by a processor, cause the processor to:
obtain at least one monopolar response of a recipient of an implantable medical device to at least one monopolar stimulation measurement performed on the recipient via the implantable medical device;
obtain one or more focused responses of the recipient of the implantable medical device to one or more focused stimulation measurements performed on the recipient via the implantable medical device, wherein the at least one monopolar response and the one or more focused responses are a same type of response obtained from the recipient;
analyze the at least one monopolar response relative to the one or more focused responses; and
based on the analyzing of the at least one monopolar response relative to the one or more focused responses, recommending one or more operational parameters for instantiation at the implantable medical device.

26. The one or more non-transitory computer readable storage media of claim 25, wherein the at least one monopolar response and the one or more focused responses are objective responses.

27. The one or more non-transitory computer readable storage media of claim 25, wherein the at least one monopolar response and the one or more focused responses are behavioral responses.

28. The one or more non-transitory computer readable storage media of claim 25, wherein the instructions operable to cause the processor to obtain one or more focused responses of the recipient further comprise instructions operable to:
initiate a plurality of focused stimulation measurements that each evoke a focused response to form a plurality of focused responses, wherein each of the plurality of focused stimulation measurements are performed with different degrees of focusing; and
obtain the plurality of focused responses.

29. The one or more non-transitory computer readable storage media of claim 25, wherein the instructions operable to cause the processor to analyze the at least one monopolar response relative to the one or more focused responses comprise instructions operable to:
estimate one or more differences in benefits provided to the recipient by monopolar stimulation in at least one monopolar stimulation measurement and benefits provided to the recipient by focused stimulation in each of the one or more focused stimulation measurements,
wherein the one or more operational parameters are recommended based on the one or more differences in benefits provided to the recipient between monopolar stimulation and focused stimulation.

30. The one or more non-transitory computer readable storage media of claim 25, wherein the instructions operable to cause the processor to analyze the at least one monopolar response relative to the one or more focused responses comprise instructions operable to:
estimate one or more differences in benefits provided to the recipient by monopolar stimulation in at least one monopolar stimulation measurement and benefits provided to the recipient by focused stimulation in each of the one or more focused stimulation measurements;
estimate differences in power consumption between the monopolar stimulation at least one monopolar stimulation measurement and the focused stimulation in each of the one or more focused stimulation measurements; and
wherein the one or more operational parameters are recommended based on the one or more differences in benefits provided to the recipient between monopolar stimulation and focused stimulation and the differences in power consumption between the monopolar stimulation at least one monopolar stimulation measurement and the focused stimulation in each of the one or more focused stimulation measurements.

31. The one or more non-transitory computer readable storage media of claim 25, wherein the instructions operable to cause the processor to recommend one or more operational parameters for instantiation at the implantable medical device comprise instructions operable to:
recommend a degree of focusing for use in delivering stimulation signals at one or more stimulation channels of the implantable medical device.

32. The one or more non-transitory computer readable storage media of claim 31, further comprising instructions operable to:
recommend use of one or more degrees of focusing for use in delivering stimulation signals at one or more stimulation channels of the implantable medical device only in certain sound environments.

33. The one or more non-transitory computer readable storage media of claim 25, wherein the instructions operable to cause the processor to recommend one or more operational parameters for instantiation at the implantable medical device comprise instructions operable to:
recommend a set of stimulation channels for use in delivering stimulation signals to the recipient of the implantable medical device.

34. The one or more non-transitory computer readable storage media of claim 25, wherein the instructions operable to cause the processor to recommend one or more operational parameters for instantiation at the implantable medical device comprise instructions operable to:
recommend current steering parameters for use in delivering stimulation signals to one or more locations between stimulation contacts of the implantable medical device.

35-39. (canceled)
